(19) United States
(12) Patent Application Publication
(10) Pub. No.: US 2014/0257104 A1
(43) Pub. Date: Sep. 11, 2014

(54) METHOD AND SYSTEM FOR ULTRASOUND IMAGING

(71) Applicant: EZONO AG, Jena (DE)

(72) Inventors: Allan DUNBAR, Jena (DE); Eliseo Sobrino, Jena (DE)

(73) Assignee: eZono AG, Jena (DE)

(21) Appl. No.: 13/786,011

(22) Filed: Mar. 5, 2013

(51) Int. Cl. A61B 8/00; A61B 8/08; A61B 8/14

(52) U.S. Cl. CPC A61B 8/4254; A61B 8/14; A61B 8/4263; A61B 8/5207; A61B 8/54; A61B 8/466; A61B 8/4427
USPC 600/443

(57) ABSTRACT

A system and method for assembling a 3D ultrasound image representation from multiple two-dimensional ultrasound images utilises a magnetic position detection system to detect the ultrasound probe position and allow mapping of the multiple two-dimensional ultrasound images into a three-dimensional frame of reference. The magnetic position detection system may use magnetic markers positioned on the subject or fixed in space around the subject. The position detection may use magnetic model fitting, look-up table, triangulation or distance measurement techniques to determine the position of the ultrasound probe relative to the magnetic markers. The ultrasound probe includes a magnetometric detector to detect the field generated by the magnetic markers.

### TECHNICAL FIELD

The present invention relates to a method and system for ultrasound imaging, and in particularly to constructing a three-dimensional representation of the internal structure of a subject.

### BACKGROUND AND OVERVIEW

Unless explicitly indicated herein, the materials described in this section are not admitted to be prior art.

Ultrasound imaging is a widely-used technique for visualising internal structures without the safety and exposure limitations of imaging using electromagnetic or ionising radiation and without the complexity of magnetic resonance imaging techniques. Its low cost and relative ease of use makes it an increasingly popular choice in medical imaging applications and its use for obstetric sonography during pregnancy is widespread.

A typical 2D B-mode ultrasound image is a greyscale image representing a cross-sectional slice through the subject. Typically the imaged slice is very thin, of the order of 1 mm, and orienting and positioning the ultrasound transducer differently on the subject allows the operator to image the internal structure of the subject in different places and from different directions. It is also known to assemble such two-dimensional slice images into a three-dimensional representation of the internal structure of the subject. The three-dimensional representation may be displayed or visualised in different ways, for example by simply displaying the different slices in a multi-slice display or by using volume rendering techniques to form a more realistic 3D image.

In order to combine the two-dimensional images together it is necessary to know their positional relationship. In other words their relative location and orientation in a common three-dimensional frame of reference needs to be established. There are generally two classes of technique for achieving this, one by detecting the position of the ultrasound probe as each of the two-dimensional slice images are acquired and the other by image analysis to identify common structures in the image and then estimating spatial transformations between them. In the position detection techniques, one example is to mount the ultrasound transducer array in the probe on an internal frame with a motor for rotating the array back and forth. The relative positional relationship of the acquired two-dimensional images can be deduced from the position of the array at the time of image acquisition and so a three-dimensional image can be constructed. Another example is to track the position of the probe using electromagnetic or optical tracking technology and, again, knowing the location and orientation of the probe associated with each two-dimensional image allows the construction of the three-dimensional image.

Mechanical mounting and moving of the ultrasound probe, however, is complex and requires the provision of accurate mounts and transducers and makes the probe larger. It also is not very reliable and has only a limited field of view. Optical and electromagnetic tracking technologies also have problems of the need for line-of-sight and the need for complicated transmitters and sensors. These therefore increase the cost and complexity of what is meant to be a simple imaging technique.

It would therefore be advantageous to have a simpler and cheaper way of constructing three-dimensional ultrasound images.

With the present invention the position (i.e. location and/or orientation) of an ultrasound transducer is tracked by means of a magnetic position detection system as two-dimensional ultrasound images are acquired. The knowledge of the positioning of the ultrasound transducer when each two-dimensional ultrasound image was acquired allows the two-dimensional ultrasound images to be assembled into a three-dimensional representation of the subject.

In more detail one embodiment of the invention provides an ultrasound imaging system comprising: an ultrasound transducer for transmitting ultrasound into a subject and receiving ultrasound echoes from the subject; a controller for controlling the ultrasound transducer and comprising a data processor for processing data representing the received echoes to construct from it a two-dimensional representation of the internal structure of the subject; a magnetic position detection system comprising a magnet and a magnetometric detector for detecting the magnetic field generated by the magnet, one of the magnet or magnetometric detector being attached to the ultrasound transducer and the other being in a reference position, the magnetic position detection system being adapted to detect the relative positioning of the magnet and magnetometric detector; wherein the data processor is adapted to construct a three-dimensional representation of the internal structure of the subject from plural two-dimensional representations taken with the ultrasound transducer different positioned by assembling the data from the plural two-dimensional representations utilising the detected relative positioning of the magnet and magnetometric detector; the system further comprising a display for displaying the three-dimensional representation.

Preferably the magnetic position detection system detects one, or more preferably both, of the spatial location and spatial orientation of the ultrasound probe, by detecting at least one, preferably both, of the relative spatial location and relative spatial orientation of the magnet and magnetometric detector. It should be noted that it is the relative position of each of the two-dimensional ultrasound images which is detected so that they can be registered, i.e. mapped, into a common three-dimensional frame of reference. Preferably the reference position is fixed in space, for example by being fixed to structure around the subject, but alternatively the reference position could be fixed on the subject. Fixing the reference position on the subject is useful in situations where the subject is in motion, for example a human or animal subject, as it allows the different two-dimensional images to be registered in the frame of reference of the subject, which is moving. This therefore can compensate for movement such as breathing.

Preferably the magnet is in the reference position and the magnetometric detector is attached to the ultrasound transducer. The magnet may be fixed in the reference position by use of an adhesive fixing such a skin-adhering patch or plaster.

Plural magnets, optionally in different orientations, can be provided to give higher accuracy of registration. The plural magnets may be in the same adhesive fixing, or in different ones.

Alternatively, the magnet may be on or an integral part of a tool for insertion into the subject, for example in the medical field a tissue-penetrating medical tool such as a needle, cannula, stylet or catheter. By holding the tool still while ultrasound imaging from different positions (i.e. locations and/or orientations), a three-dimensional representation can be constructed.

The magnet is preferably a permanent magnet, though an electromagnet can be used. The magnetic position detection system may utilise any suitable techniques such as magnetic field model fitting, use of a look-up table representing magnetic field values, measuring the distance between the magnet and the magnetometric detector or triangulation.

Preferably the ultrasound transducer is a freehand, i.e. handheld, transducer with the magnetometric detector attached to it.

The three-dimensional representation may be displayed in multi-slice or volume-rendered format as desired.

The system is particularly suitable for three-dimensional imaging of a human or animal subject, but can also be used in other ultrasound imaging fields.

### BRIEF DESCRIPTION OF THE DRAWINGS

The invention will be further described by way of example with reference to the accompanying drawings in which:

FIG. 1 schematically illustrates an ultrasound imaging system in accordance with one embodiment of the present invention;

FIG. 2 schematically illustrates in block diagram form the magnetometric detector used in the embodiment of FIG. 1;

FIG. 3 schematically illustrates in block diagram 4 the magnetometric detector base station used in the embodiment of FIG. 1; and

FIG. 4 is a flow diagram explaining a 3D ultrasound imaging method in accordance with an embodiment of the invention.

FIG. 5 schematically illustrates a magnetic marker in accordance with one embodiment of the invention; and

FIG. 6 schematically illustrates a magnetic marker according to another embodiment of the invention.

### DETAILED DESCRIPTION

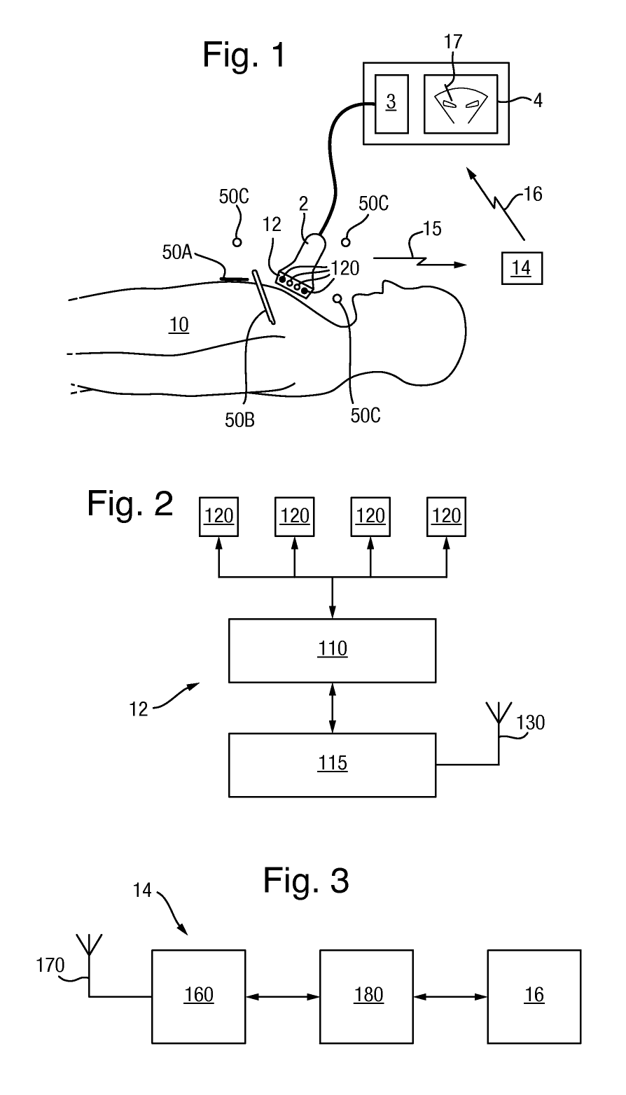

As shown in FIG. 1 the system in this embodiment of the invention comprises an ultrasound imaging system I including an ultrasound transducer 2, system processor 3 and display 4. The system also comprises a magnetic marker or markers 50A, B or C which form reference points for the 3D image construction process.

To detect the position of the magnetic marker or markers 50A, B or C, the ultrasound transducer 2 is provided with a magnetometric detector 12 comprising an array of magnetometers 120. The detector 12 senses the magnetic field from the magnetic marker or markers 50A, B or C, together with the terrestrial magnetic field and any other background magnetic field, and the processor 3 is adapted to determine from the detected field the location and orientation of the magnetometric detector 120 relative to the magnetic marker or markers 50A, B or C. This magnetically detected position is then associated with the 2D ultrasound image acquired with the ultrasound transducer in that position.

The ultrasound system 1 can be a standard two-dimensional B-mode ultrasound system with the standard ultrasound probe 2 being modified by the provision of the magnetometric detector 12. The processor 4, which is connected to the ultrasound probe 2 via a cable, drives the ultrasound transducer 2 by sending electrical signals to cause it to generate ultrasound pulses and interpreting the raw data received from the transducer 2, which represents echoes from the subject's body 10, to assemble it into a 2D image of the patient's tissue.

The magnetometric detector 12 may be detachably attached to the ultrasound transducer 2 and can be battery-powered or powered from the ultrasound system. Preferably positioning elements are provided on the magnetometric detector 12 to ensure that it is always attached in the same well-defined position and orientation. The magnetometric detector 12 is connected by a wireless connection 15 to a base unit 14 which is in wireless or wired (e.g. USB) communication 16 with the ultrasound system processor 3 and display 4. The base unit 14 can be integrated with, or some of its functions performed by, the ultrasound system processor 3 or the magnetometric detector 12. As will be explained in more detail below, the base unit 14 receives normalised measurements from magnetometric detector 12 and calculates the position, i.e. location and orientation, relative to the magnetic marker or markers 50A, B or C. The base unit 14 can also receive additional information such as the state of charge of the magnetometric detector's battery and information can be sent from the base unit 14 to the magnetometric detector 12, such as configuration information. The base unit 14 forwards the results of its calculations, i.e. the relative position of the magnetometric detector 12 and the magnetic marker or markers 50A, B or C to the ultrasound image processor 3 to allow it to assemble the 3D representation. This will be explained in more detail below.

FIG. 1 schematically illustrates three different forms of magnetic marker 50A, 50B and 50C. The marker 50A is an elongate permanently magnetised element carried by an adhesive patch or plaster. Such a marker is illustrated schematically in plan view in FIG. 5. It comprises a skin-adhering patch or sheet 500 which has a lower adhesive layer and an upper protective layer and optionally intermediate layers. Between the layers an elongate permanently magnetised element 502 is encapsulated. The element 502 may be of any magnetic material such as steel or stainless steel of similar gauge to a hypodermic syringe or a wire containing iron or another magnetic material. Alternatively a magnetic substance may be deposited in a line or other pattern on one of the layers. The sheet 500 may be a conventional plaster or skin patch containing the magnetic element 502.

FIG. 6 shows an alternative embodiment of the magnetic marker 50A in which a plurality, for example 2, elongate magnets 504 are positioned. Again these may be metallic, e.g. steel, elements which have been magnetised, or can be deposits of magnetic material. As indicated in FIG. 6 the orientation of the two elements 504 is different, this providing more accuracy in the position detection process.

Although not illustrated in FIG. 1, plural markers 50A as exemplified by FIG. 5 or FIG. 6 can be attached to the skin of the patient in different locations and orientations to improve the accuracy of the position detection process.

FIG. 1 schematically illustrates as 50B an alternative form of magnetic marker which is a tissue-penetrating medical tool such as a needle, stylet, cannula or catheter. Such a tool can either be magnetised itself if of suitable material, or can carry permanent magnets or electromagnets. If such a tool is used as a marker for the 3D image construction process it is necessary that its position is not changed from image to image. Thus the tool 50B would be held steadily in position while the ultrasound probe 2 is moved to different positions (locations and/orientations) to acquire the plural 2D images which are then assembled or mapped into a common 3D frame of reference using the detected position of the tool 50B as a reference.

FIG. 1 also schematically illustrates a third alternative form of magnetic marker 50C in which individual magnets are positioned at fixed positions in space around the subject 10. Such markers 50C can be simple permanent magnets which are adhesively attached to fixed locations around the subject, for example the bed or table on which the subject is supported, a frame or other structure near the subject or surrounding walls or furniture. The only requirement is that the markers remain in a position which is fixed as the 2D images are acquired so that they provide a consistent reference point for the assembling or mapping of the 2D images into a common 3D frame of reference. The markers 50C can be adhesive patches including elongate elements as illustrated in FIGS. 5 and 6.

A magnetized needle of around 4 cm length has a range of up to 4 cm in terms of accurate position detection using the modelling technique discussed below as beyond this we are at the noise limit of the sensors. Using an elongated cylinder of highly magnetic material would give a higher position range because of the higher magnetic field and thus stringer “signal”. Rare earth magnets can generate fields over 100 times stronger than can steel and, when the marker is not doubling-up as a tool, significantly more material can be used to construct the marker compared to a tool such as a standard needle. Thus the functional range can be increased significantly using strong magnets as markers. In practice, high field strengths can saturate the sensors used. Therefore this would limit use in the near field of such a marker. So there is a functional range between two concentric circles around the or each marker. Nevertheless this gives a clinically-useful sized working areas on patients, for example 5 to 15 centimetre areas on the skin. Optionally the marker may be within a patch which has the clinically useful range, or at least the inner bound, marked on it using boundary markings or coloured areas to assist the clinician in locating the markers to give good results. Alternatively the patch may be a circular patch whose radius indicates the inner limit.

An important point about the positioning of the magnetic marker 50A on the body of the subject is that the marker will move with the subject. This can be advantageous as it provides a self-compensation for the normal movement, e.g. respiration, of the subject allowing the various 2D images to be assembled to form a 3D representation in the (moving) frame of reference of the subject. It will be appreciated that with the markers 50C that are fixed in space, movement of the subject between image acquisitions will result in misregistration of the 2D images and thus a poor 3D representation.

The magnetometric detector 12 and example ways in which the position of the ultrasound probe 2 is calculated will now be explained in more detail. Similar techniques are described in our co-pending International (PCT) patent application PCT/EP2011/065420.

The components of the magnetometric detector 12 are shown schematically in greater detail in the block diagram of FIG. 2. The magnetometric detector 12 comprises an array of two or more (e.g. four) magnetometers 120 (not shown in FIG. 2) whose outputs are sampled by a microprocessor 110. The microprocessor 110 normalizes the measurement results obtained from the magnetometer array 100 and forwards it to a transceiver 115 with an antenna 130 which, in turn transmits the information to the base unit 14. In a modified version of this embodiment, the magnetometric detector 12 is provided with a multiplexer rather than with a microprocessor 110 and the normalization is performed by a processor 180 in the base unit 14.

Each magnetometer 120 in the array 100 of magnetometers measures the components aₖ^{u}, aₖ^{v},aₖ^{w} (k indicating the respective magnetometer) of the magnetic field at the position of the respective magnetometer 120 in three linearly independent directions. The microprocessor 110 transforms these raw values:

aₖ=(aₖ^{u},aₖ^{v},aₖ^{w})

into corresponding normalized values:

bₖ=(bₖ^{u},bₖ^{v},bₖ^{w})

in predetermined orthogonal directions of equal gain by multiplying the three values aₖ obtained from the magnetometer with a normalisation matrix Mₖ and adding a normalisation offset vector βₖ:

bₖ=aₖ*Mₖ+βₖ

as will be described in more detail below. The normalisation matrices and the normalisation offset vectors are permanently stored in a memory associated with the microcontroller. This same transformation is performed for each of the magnetometers 120 with their respective normalisation matrix and adding a normalisation offset vector such that the result bₖ, for each magnetometer provides the components of the magnetic field in the same orthogonal spatial directions with identical gain. Thus, in a homogenous magnetic field, all magnetometers always provide identical values after normalisation regardless of the strength or orientation of the homogenous magnetic field.

Normalisation and Offset

All magnetometers should measure equal values when exposed to a homogeneous field. For example, a magnetometer rotated in the homogeneous terrestrial magnetic field should, depending on the orientation of the magnetometer, measure varying strengths of the components of the magnetic field in the three linearly independent directions. The total strength of the field, however, should remain constant regardless of the magnetometer's orientation. Yet, in magnetometers available on the market, gains and offsets differ in each of the three directions. Moreover, the directions often are not orthogonal to each other. As described, for example, in U.S. Pat. No. 7,275,008 B2, for a single sensor, if a magnetometer is rotated in a homogeneous and constant magnetic field, the measurements will yield a tilted 3-dimensional ellipsoid. Because the measured field is constant, however, the normalized measurements should lie on a sphere. Preferably, an offset value β and a gain matrix M are introduced to transform the ellipsoid into a sphere.

With a set of sensors, additional steps need to be taken to assure that the measurements of different sensors are identical with each other. To correct this, preferably, set of a gain normalisation matrices Mₖ and normalisation offset vectors βₖ for each position k are determined which transform the magnetometer's raw results aₖ into a normalized result bₖ:

bₖ=aₖ*Mₖ+βₖ

Such a set of gain matrices Mₖ can be obtained by known procedures, for example the iterative calibration scheme described in Dorveaux et. al., “On-the-field Calibration of an Array of Sensors”, 2010 American Control Conference, Baltimore 2010.

By virtue of the defined transformation, bₖ provides the strength of the component of the magnetic field in three orthogonal spatial directions with equal gain. Moreover, it is ensured that these directions are the same for all magnetometers in the magnetometric detector. As a result, in any homogeneous magnetic field, all magnetometers yield essentially identical values.

The normalisation information Mₖ and βₖ for each magnetometer as obtained in the calibration step can be stored either in the magnetometric detector 12 itself or in the base unit 14. Storing the information in the magnetometric detector 12 is preferred as this allows easy exchange of the magnetometric detector 12 without the need to update the information in the base unit. Thus, in a preferred embodiment of the invention, the outputs of the magnetometers of the magnetometric device are sampled and their results are normalised in the magnetometric detector 12. This information, together with any other relevant information, is transmitted to the base unit 14 for further analysis.

In another embodiment of the invention, the transformation can be another, more general non-linear transformation bₖ=F(aₖ).

In addition to the above calibration method, another calibration method is applied in this embodiment which employs an inhomogeneous magnetic field to obtain the relative spatial locations of the magnetometric detector's magnetometers. While standard calibration methods utilize a homogenous magnetic field to (a) align the measurement axis of the magnetometers orthogonally, (b) cancel the offset values and (c) adjust to equal gain, it is of further advantage that also the precise relative spatial locations of the magnetometers are available. This can be achieved by an additional calibration step in which the magnetometric detector is subjected to a known inhomogeneous magnetic field. Preferably, comparing the obtained measurements at the various positions to the expected field strengths and/or orientations in the assumed locations, and correcting the assumed locations until real measurements and expected measurements are in agreement, allows for the exact calibration of the spatial positions of the sensors.

In a variation of the latter calibration method, an unknown rather than a known homogeneous field is used. The magnetometers are swept through the unknown magnetic field at varying positions, with a fixed orientation. With one of the magnetometers supplying a reference track, the positions of the other magnetometers are adaptively varied in such a way that their measurements align with the measurements of the reference unit. This can be achieved for example by a feedback loop realizing a mechano-magnetic-electronical gradient-descent algorithm. The tracks used in this inhomogeneous field calibration can be composed of just a single point in space.

Position Detection

The base station 14 shown schematically in greater detail in FIG. 3 receives the normalised positional information from the magnetometric detector 12 through its receiver 160 with antenna 170 and forwards the information to a processor 180. There, the normalized results of the measurements are combined to derive the position (location and orientation) of the magnetometric detector 12 relative to the magnetic marker or markers 50A, B or C.

There are various ways in which this can be done. One example is to create and store a look-up table by measuring the magnetometric detector's responses in an array of locations and orientations in the field of the magnetic marker or markers 50A, B or C. Then the position associated with each 2D image acquisition can be obtained by reading it from the look-up table using the measured field values at the time of acquisition.

Alternatively where three or more magnetic markers 50C are provided their distance and/or direction can be used to triangulate the position of the magnetometric detector 12.

In a different embodiment a model fitting process based on fitting a mathematical model of the expected field to the measurements can be used as will now be explained in detail. This model is for an elongate element 50B; different models would be needed for different shaped markers such as 50A or 50C. Where plural markers are used the model can just be the sum of the fields from the plural markers.

Model fitting

The values bₖ could be used to fit a model cₖ(p) of the combined magnetic field originating from the magnetic marker or markers 50A, B or C and the terrestrial magnetic field. The unknown parameters p in this model are the position I relative to the magnetic marker or markers 50A, B or C, and possibly the dimensions and orientation d and the magnetisation m of the magnetic marker or markers 50A, B or C, as well as the terrestrial magnetic field E:

p={I, d, m, E}

While it is possible to fit the model to the values bₖ, in this embodiment the values bₖ are converted into what we will call “gradient” values, which are deviations from an average. To calculate the average for this purpose the sensor with the largest deviation from the average over all sensors is also excluded, and any sensor which indicates saturation in any of its field components is also excluded. The mean of the remaining \tilde{k} sensors is then calculated and the gradient values for each sensor are calculated as:

Gₖ(tₗ)=b_{\tilde{k}}(tₗ)−\tilde{bₖ(tₗ)}

The model used in this embodiment models these gradient values. Thus the model cₖ(p) comprises the normalized components cₖ^{x}(p), cₖ^{y}(p), cₖ^{z}(p) of the gradient values at the position of magnetometer k at a given set of parameters p. By means of appropriate algorithms known to the skilled person the parameters p are obtained at which the sum of the squares of the residuals Rₖ, i.e. the deviation of the components of the magnetic field according to the model from the components actually measured:

ΣₖRₖ²=Σₖ(Gₖ−cₖ(p))²

is minimized or below a defined. Suitable minimization techniques are for example gradient-descent algorithms as well as Levenberg-Marquardt approaches. Moreover, Kalman filter techniques or similar iterative means can be utilized to continuously perform such an operation.

The form of the model of the magnetic field depends on the type of magnetic marker or markers 50A, B or C.

As mentioned above, one example of a suitable marker is an elongate magnetised element 50A or B similar to a standard hypodermic needle. If the needle 50A or B is sufficiently rigid, i.e. it bends only slightly, it can be approximated as a straight hollow cylinder. The magnetic field of such cylinder is equivalent to that of opposite magnetic charges (i.e. displaying opposite magnetic force) evenly distributed on the end surfaces of the cylinder, i.e. two circular rings at the opposite ends of the tools, the rings having opposite magnetic charge. In view of the small diameter of the needle 50A or B, the charges can be further approximated by two magnetic point charges (monopoles) at the opposite ends. Thus, according to the model, the magnetic field of an elongate marker 50A or B extending along the vector d measured from a position rₖ is:

cₖ(p)=m*(rₖ/|rₖ|³−(rₖ+d)/|rₖ+d|³).

Here |rₖ| and |rₖ+d| indicate the absolute values of the vectors rₖ and rₖ+d, respectively. The positions rₖ can be converted to the location I of the ultrasound transducer 2 with the help of the known positions of the magnetometers 120 in the magnetometric detector 12 and the position of the magnetometric detector 12 relatively to the ultrasound transducer 2. Note that in contrast to many known approaches the above model does not assume the field to be a dipole field. This would be an oversimplification as the magnetometric detectors in general are too close to the marker as compared to its length to make a dipole field a valid approximation.

The solution obtained by non-linear optimisation can be checked to give more confidence that it represents a true tool position. For example the values returned by the fitted model for the length of the marker and/or its magnetisation can be checked against the expected values. A length tolerance and magnetisation tolerance are defined for satisfying these tests—for example requiring solutions which have no greater than twice the actual length or magnetization will throw most of the poor solutions out but allow a solution to converge.

The relative position obtained by fitting the model to the measured gradient values Gₖ as described above is then forwarded via link 16 to the processing unit 3. There, it is associated with the acquired 2D image.

It will be appreciated that the magnetic position detection system returns the position of the magnetometric detector 12 relative to the magnetic marker or markers 50A, B or C. Because the magnetometric detector is fixed to the ultrasound probe, the relationship between the position (i.e. location with respect to three orthogonal coordinate axes x, y, z and angular orientation with respect to three axes of rotation θ, φ, ψ) of the magnetometric detector 12 and the position of the ultrasound probe 2 P_{probe}(x,y,z, θ, φ, ψ) is fixed. The relationship between the 2D ultrasound image and the probe is also fixed (or known in the case of probes that can move the ultrasound beam), and thus the data constituting the 2D images can be expressed as values of intensity as a function of positions in 2 dimensions (x′,y′) relative to the probe. These positions can be mapped by linear transformations into a single, common 3D frame of reference based on the magnetically detected probe location and orientation.

The positions of the intensities in the common 3D frame of reference are:

r=T+Rr′

where:
r=(x,y,z) is the position the intensity in the common 3D frame of reference

r′=(x′,y′,0)
T=(X,Y,Z) is a translation transform where (X,Y,Z) is the estimated position of the probe in the common 3D frame of reference
R=is the three-dimensional rotation transform matrix using θ, φ, ψ which are the estimated orientation of the probe relative to the common 3D frame of reference

$$\begin{matrix}R = {R}_{z} ( ψ ) {R}_{y} ( θ ) {R}_{x} ( φ ) \\ = [ \begin{matrix}cos θcos ψ & - cos φ sin ψ + sin φ sin θ cos ψ & sin φ sin ψ + cos φ sin θ cos ψ \\ cos θsin ψ & cos φ cos ψ + sin φ sin θ sin ψ & - sin φ cos ψ + cos φ sin θ sin ψ \\ - sin θ & sin φ cos θ & cos φ cos θ\end{matrix} ]\end{matrix}$$

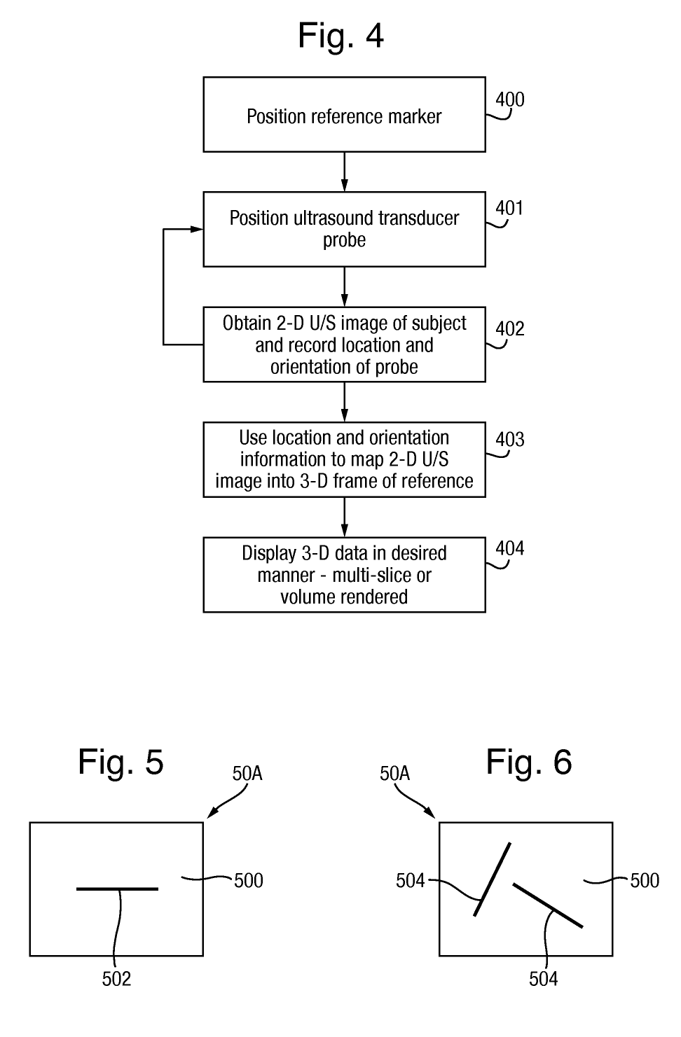

FIG. 4 illustrates the whole process of obtaining a 3D ultrasound image. In step 400 the reference marker or markers 50A, B or C are positioned as desired, either on the subject or in the space around the subject. Then in step 401 the ultrasound probe 2 is positioned to image the desired internal structure of the subject and in step 402 a 2D ultrasound image is obtained in that position. The position, i.e. location and orientation of the ultrasound probe 2 as measured by the magnetic position detection system are also recorded associated with the 2D ultrasound image. The ultrasound transducer probe is then repositioned, i.e. its location and/or its orientation are changed to acquire a different image of the internal structure associated with the different location and/or orientation of the probe. Steps 401 and 402 can be repeated any desired number of times.

In step 403 each 2D image is mapped into the three-dimensional frame of reference defined by the magnetic markers 50A, B and C using the location and orientation information. Then in step 404 the ultrasound image data in the three-dimensional frame of reference is displayed to the user in the desired manner—either as multiple slices or optionally volume-rendered.

It should also be noted that by leaving adhesive markers 50A on the subject over a period of time, it is possible for 3D images assembled on different occasions to be compared, which could be advantageous in monitoring anatomical changes, such as tumour growth, changing organ size, healing processes etc.

The embodiment described above utilises the magnetic markers to derive both the location and orientation information. However the ultrasound probe can include an inertial position measurement unit using gyroscopic components to detect the movement of the probe from an initial location and/or orientation. For example, gyroscopic sensors can be used to measure the orientation of the probe with the location being detected by the magnetic position detector. Alternatively either or both of the location and orientation can be measured by both systems and the results fused to provide better estimates.

In alternative embodiment, not illustrated, the ultrasound probe 2 is provided with one or more magnets and the field from these magnets is detected by magnetometric detectors positioned either on the subject or in the space around the subject. Thus the position of the magnetometric detectors and magnets are reversed as compared with the illustration of FIG. 1. As all that is required is the relative position of the magnet and magnetometric detectors, the same position detection techniques can be used as explained above. However this alternative arrangement allows a relatively simple modification to the ultrasound probe 2 (namely adding one or more permanent magnets), while magnetic sensors can be positioned in the space around the subject.

1. An ultrasound imaging system comprising:
an ultrasound transducer for transmitting ultrasound into a subject and receiving ultrasound echoes from the subject;
a controller for controlling the ultrasound transducer and comprising a data processor for processing data representing the received echoes to construct from it a two-dimensional representation of the internal structure of the subject;
a magnetic position detection system comprising a magnet and a magnetometric detector for detecting the magnetic field generated by the magnet, one of the magnet or magnetometric detectors being attached to the ultrasound transducer and the other being in a reference position, the magnetic position detection system being adapted to detect the relative positioning of the magnet and magnetometric detector;
wherein the data processor is adapted to construct a three-dimensional representation of the internal structure of the subject from plural two-dimensional representations taken with the ultrasound transducer different positioned by assembling the data from the plural two-dimensional representations utilising the detected relative positioning of the magnet and magnetometric detector; and
the system further comprising a display for displaying the three-dimensional representation.

2. The ultrasound imaging system according to claim 1, wherein the magnetic position detection system is adapted to detect as the relative positioning at least one of the relative spatial location and relative spatial orientation of the magnet and magnetometric detector.

3. The ultrasound imaging system according to claim 1, wherein the magnetic position detection system is adapted to detect as the relative positioning both of the relative spatial location and relative spatial orientation of the magnet and magnetometric detector.

4. The ultrasound imaging system according to claim 1, wherein the ultrasound transducer is a freehand handheld ultrasound transducer.

5. The ultrasound imaging system according to claim I, wherein utilising the detected relative positioning of the magnet and magnetometric detector comprises registering the plural two-dimensional representations to a common frame of reference for the three-dimensional representation.

6. The ultrasound imaging system according to claim 1, wherein the reference position is fixed in space.

7. The ultrasound imaging system according to claim 1, wherein the reference position is fixed on the subject.

8. The ultrasound imaging system according to claim 1, wherein the magnet is in the reference position and the magnetometric detector is attached to the ultrasound transducer.

9. The ultrasound imaging system according to claim 8, wherein the magnet is held by an adhesive fixing.

10. The ultrasound imaging system according to claim 9, wherein the adhesive fixing is a skin-adhering patch carrying the magnet.

11. The ultrasound imaging system according to claim 1, wherein plural magnets are provided.

12. The ultrasound imaging system according to claim 1, wherein plural magnets are provided in different orientations.

13. The ultrasound imaging system according to claim 1, wherein the magnet is provided on an element for insertion into the interior of the subject.

14. The ultrasound imaging system according to claim 1, wherein the magnet is one of a permanent magnet, and an electromagnet

15. The ultrasound imaging system according to claim 1, wherein the display is adapted to display the three-dimensional representation as a volume-rendered representation.

16. The ultrasound imaging system according to claim 1, wherein the display is adapted to display the three-dimensional representation as a multi-slice representation.

17. The ultrasound imaging system according to claim 1, wherein the magnetic position detection system is adapted to detect the relative positioning of the magnet and magnetometric detector by one of: magnetic field model fitting, use of a look-up table representing magnetic field values, measuring the distance between the magnet and the magnetometric detector and triangulation.

18. The ultrasound imaging system according to claim 1, wherein the subject is human or animal and the data processor is adapted to construct a three-dimensional representation of the internal anatomy of the subject.

19. A method of forming a three-dimensional representation of the internal structure of a subject comprising the steps of:
transmitting ultrasound into a subject and receiving ultrasound echoes from the subject using an ultrasound transducer a plurality of times with the ultrasound transducer differently positioned relative to the subject;
processing data representing the received echoes to construct from plural two-dimensional representations of the internal structure of the subject;
detecting the different positioning of the ultrasound transducer relative to the subject with a magnetic position detection system comprising a magnet and a magnetometric detector for detecting the magnetic field generated by the magnet, one of the magnet or magnetometric detector being attached to the ultrasound transducer and the other being in a reference position;
constructing a three-dimensional representation of the internal structure of the subject from the plural two-dimensional representations by assembling the data from the plural two-dimensional representations utilising the detected positioning of the ultrasound transducer relative to the subject; and
displaying the three-dimensional representation.

20. The method according to claim 19, comprising the step of detecting as the relative positioning at least one of the relative spatial location and relative spatial orientation of the magnet and magnetometric detector.

21. The method according to claim 19, comprising the step of detecting as the relative positioning both of the relative spatial location and relative spatial orientation of the magnet and magnetometric detector.

22. The method according to claim 19, wherein the ultrasound transducer is a freehand handheld ultrasound transducer.

23. The method according to claim 19, comprising the step of registering the plural two-dimensional representations to a common frame of reference for the three-dimensional representation.

24. The method according to claim 19, wherein the reference position is fixed in space.

25. The method according to claim 19, wherein the reference position is fixed on the subject.

26. The method according to claim 19, wherein the magnet is in the reference position and the magnetometric detector is attached to the ultrasound transducer.

27. The method according to claim 26, wherein the magnet is held by an adhesive fixing.

28. The method according to claim 27, wherein the adhesive fixing is a skin-adhering patch carrying the magnet.

29. The method according to claim 19, comprising the step of providing plural magnets.

30. The method according to claim 29, wherein the plural magnets are provided in different orientations.

31. The method according to claim 19, comprising the step of providing the magnet on an element for insertion into the interior of the subject.

32. The method according to claim 19, wherein the magnet is one of: a permanent magnet, an electromagnet.

33. The method according to claim 19, wherein the step of displaying the three-dimensional representation comprises displaying a volume-rendered representation.

34. The method according to claim 19, wherein the step of displaying the three-dimensional representation comprises displaying a multi-slice representation.

35. The method according to claim 19, wherein the magnetic position detection system is adapted to detect the relative positioning of the magnet and magnetometric detector by one of magnetic field model fitting, use of a look-up table representing magnetic field values, measuring the distance between the magnet and the magnetometric detector and triangulation.

36. The method according to claim 19, wherein the subject is human or animal and the method comprises constructing a three-dimensional representation of the internal anatomy of the subject.
